(19) United States
(12) Patent Application Publication
(10) Pub. No.: US 2026/0249569 A1
(43) Pub. Date: Aug. 27, 2026

(54) A PREPREG, A METHOD OF TESTING THEREOF AND A PROCESS FOR ITS MANUFACTURE

(71) Applicant: HEXCEL COMPOSITES LIMITED, Duxford (GB)

(72) Inventors: James LINDSAY, Impington (GB); Andrea CABALLERO, Baldock (GB); Marco ARCIDIACONO, Duxford (GB)

(73) Assignee: HEXCEL COMPOSITES LIMITED, Duxford (GB)

(21) Appl. No.: 18/871,269

(22) PCT Filed: Jun. 1, 2023
(86) PCT No.: PCT/GB2023/051445

§ 371 Date: Dec. 3, 2024

(30) Foreign Application Priority Data
Jun. 7, 2023 (GB) 2208292.9

(51) Int. Cl. B29C 70/54; B29C 70/20; B29K 101/10; B29K 307/04; B29K 309/08; G01N 3/08; G01N 5/00; G01N 33/00

(52) U.S. Cl. CPC B29C 70/54; B29C 70/20; B29K 2101/10; B29K 2307/04; B29K 2309/08; G01N 3/08; G01N 5/00; G01N 33/0078

(57) ABSTRACT

A prepreg comprising a structural layer comprising fibres having interstices therebetween, and comprising curable thermosetting resin impregnated within the structural layer and present within the interstices, wherein the prepreg comprises a test region that is free of curable thermosetting resin; a method of testing the pregreg, wherein the method comprises the steps of a) carrying out a measurement test on a test area of the test region, followed by b) inferring a property of the prepreg from the results of the measurement; and a process for the manufacture of the prepreg, the process comprising the steps of: providing a structural layer comprising fibres, having a first face and a second face, and a first impregnating layer comprising curable thermosetting resin; bringing the first face of the structural layer into contact with the first impregnating layer; compressing the structural layer and first impregnation layer together so that curable resin impregnates the fibrous layer so that it is present between the interstices between the fibres, thereby forming the prepreg; wherein the first impregnating layer comprises a region where no impregnation occurs, thereby producing the test region of the prepreg that is free of curable resin.

### TECHNICAL FIELD

The present invention relates to a prepreg comprising a test region, particularly, but not exclusively, to enable the accurate measurement of the fibre areal weight of the prepreg, a method of testing carried out on the test region and a process of manufacturing the prepreg.

### BACKGROUND

Composite materials have well-documented advantages over traditional construction materials, particularly in providing excellent mechanical properties at very low material densities. As a result, the use of such materials is widely used and their fields of application range from “industrial” and “sports and leisure” to high performance aerospace components.

Prepregs, comprising a fibre or fabric arrangement impregnated with thermosetting resin such as epoxy resin, are widely used in the generation of such composite materials. The resin may be combined with the fibres or fabric in various ways. The resin may be tacked to the surface of the fibrous material, however more usually it partially or completely impregnates the interstices between the fibres. Although it is generally known how much fibre and resin is introduced into the pregreg during its manufacture, the process of manufacture induces physical and chemical changes that make it necessary to carry out testing of the formed prepreg in order to determine its physical properties.

Various methods have been proposed for the production of prepregs, one of the preferred methods being the impregnation of a moving fibrous web with a liquid, molten or semi-solid uncured thermosetting resin.

Once manufactured, typically a number of plies of such prepregs are “laid-up” as desired and the resulting prepreg stack, i.e. a laminate or preform, is cured, typically by exposure to elevated temperatures, to produce a cured composite structure.

Curing may be performed in a vacuum bag which may be placed in a mould for curing. Alternatively the stack may be formed and cured directly in a mould.

However, prior to curing, a variety of quality control measurements are typically carried out on the pregreg, in order to ensure that it meets the requirements for its eventual application. One fundamentally important measurement is to accurately determine the relative weights of resin and fibres in the prepreg. The amount of fibres is defined as ‘fibre areal weight’ (FAW), which is the weight of fibres per unit area of the prepreg, not including the impregnated resin. The amount of resin is defined as the ‘resin content’ (RC), which is the weight of resin per unit area of the prepreg. As the prepreg has fibres impregnated with resin, these are difficult measurements to make. As such, various methods have been devised to calculate this.

One commonly employed method of measuring FAW and RC is the ‘washout’ test. This test involves taking a 100 cm² sample of the prepreg, weighing the sample, then using solvent (e.g. methylene chloride) to remove any resin from the sample. The sample is then placed in an oven to remove the solvent and the sample is weighed again to find the FAW. The FAW can then be subtracted from the total weight to infer the RC. Whilst this method is sufficiently accurate for quality control purposes, it suffers from various drawbacks. The method is set out in ASTM D3529.

The solvents used, especially methylene chloride, present a number of environmental, health and safety concerns and hazards. Additionally, experience shows that despite care being taken there is always a small amount of resin that is not removed by the solvent, and there has always been a suspicion that some of the sizing on the fibres can get removed by the solvent, which can result in inaccuracies appearing in the test method.

Alternatives to the washout method have therefore been developed, such as the Eddycus™ CF Inline FAW testing solution provided by Suragus, which uses a sensor utilising eddy current technology to measure the FAW of carbon fibres.

Therefore improvements in this area would be highly desirable.

### SUMMARY OF INVENTION

In a first aspect, the invention relates to prepreg comprising a structural layer comprising fibres having interstices therebetween, and comprising curable thermosetting resin impregnated within the structural layer and present within the interstices, wherein the prepreg comprises a test region that is free of curable thermosetting resin.

Thus, the test region of the prepreg is made only of the fibres and is completely free of curable resin, and so none needs to be removed in order to carry out tests on the fibres of the prepreg. Tests may be therefore be carried out on the test region, e.g. to determine the fibre areal weight (FAW) in a convenient and accurate manner.

The term “free of curable resin” means that there is no measurable amount of curable resin present. This does not include any ‘sizing’ that may be present on the surface of certain fibre types, present as a result of the fibre manufacture.

The test region of the prepreg may adopt a number of different sizes, depending on the type of testing envisaged. Preferably the test region has a surface area of from 1 to 1000 cm², preferably from 50 to 500 cm²., more preferably from 50 to 200 cm². In general, the larger the patch, the more waste of the prepreg is generated, however potentially a more accurate sampling of the prepreg may be achieved. A convenient balance between these has resulted in a convenient size of 100 cm².

In order for the test region to more accurately represent the prepreg as a whole it is preferable that the test region is surrounded by regions of the prepreg that are impregnated with curable thermosetting resin.

Preferably, the prepreg may comprise a second test region that is free of curable thermosetting resin. They may also comprise a third test region that is free of curable thermosetting resin. Having multiple test regions can be particularly useful when the testing is destructive, or when testing multiple regions of the prepreg is desirable. Therefore, preferably, if there are multiple test regions, these are clustered together to form a test cluster region, in which they are spaced apart such that each test region samples a different region of the prepreg in the test cluster. Advantageously, the test regions are spaced along the width of the prepreg, which is particularly useful when the fibres are unidirectional and parallel to the length of the prepreg, so that sampling of different unidirectional fibres across the width is possible.

The prepreg is typically produced as a continuous web of material, as discussed below, having a length greater than its width, typically much greater. Such prepregs are generally produced as a prepreg roll, the length of which is given by the width of the prepreg. In view of the tacky nature of the prepreg, a backing sheet is generally provided to enable the prepreg roll to be unfurled at the point of use.

The fibres may be in the form of a fabric or be formed from tows of discrete fibres. However, in general a fabric will be more resistant to physical alterations resulting from the prepreg formation process. The present invention is therefore more usefully applied when the fibres are discrete and not interwoven. Preferably the fibres are unidirectional, in that they are arranged parallel to each other, and in general will be parallel to the length of the prepreg. The fibres may comprise cracked (i.e. stretch-broken), selectively discontinuous or continuous fibres.

The fibres may be made from a wide variety of materials, such as carbon, glass, graphite, metallised polymers, metal-coated fibres and mixtures thereof. Carbon and glass fibres are preferred.

Typically the fibres in the structural layer will generally have a circular or almost circular cross-section with a diameter in the range of from 3 to 20 μm, preferably from 5 to 12 μm.

Exemplary layers of unidirectional fibres are made from HexTow™ carbon fibres, which are available from Hexcel Corporation. Suitable HexTow™ carbon fibres for use in making many unidirectional fibre layers include: IM7 carbon fibres, which are available as fibres that contain 6,000 or 12,000 filaments and weigh 0.223 g/m and 0.446 g/m respectively; IM8-IM10 carbon fibres, which are available as fibres that contain 12,000 filaments and weigh from 0.446 g/m to 0.324 g/m; and AS7 carbon fibres, which are available in fibres that contain 12,000 filaments and weigh 0.800 g/m.

The prepreg of the present invention is predominantly composed of thermosetting resin and structural fibres, although other materials are often present such as curing agents or other additives. Typically the prepregs comprise from 25 to 50 wt % of curable resin. Additionally the prepregs typically comprise from 45 to 75 wt % of structural fibres.

The resin comprises a thermosetting resin and may be selected from those conventionally known in the art, such as resins of phenol formaldehyde, urea-formaldehyde, 1,3,5-triazine-2,4,6-triamine (Melamine), Bismalemide, epoxy resins, vinyl ester resins, Benzoxazine resins, polyesters, unsaturated polyesters, Cyanate ester resins, or mixtures thereof. Epoxy resins are particularly preferred. Curing agents and optionally accelerators may be included as desired.

The thermosetting resins are preferably epoxy resins, and may comprises one or more monofunctional, difunctional, trifunctional and/or tetrafunctional epoxy resins. Such resins may become brittle upon curing, and therefore toughening materials may be included in the resin to impart durability, although this may also increase the viscosity of the resin. The toughening material may be provided as a separate layer such as a veil.

Where the toughening material is a thermoplastic polymer, it should be insoluble in the resin at room temperature and at the elevated temperatures at which the resin cures. Depending on the melting point of the thermoplastic polymer, it may melt or soften to varying degrees during curing of the resin at elevated temperatures and re-solidify as the cured laminate is cooled. Suitable thermoplastics should not dissolve in the resin, and include thermoplastics such as polyamides (PAS), polyethersulphone (PES) and polyetherimide (PEI). Polyamides such as nylon 6 (PA6), nylon 11 (PA11) or nylon 12 (PA12) and/or mixtures thereof are preferred.

The test region(s) may be usefully employed to carry out a variety of tests on the prepreg, where it is advantageous that there is no resin present. These tests could include, for example:
Thickness measurement of the test region
Damage of fibres through the process
   Signs of fuzz
   Content of broken filaments
   Or other signs of damaged fibres
Measure spreading of the fibres and look for gaps
Measure areal weight uniformity.
Take individual tow tension measurements from test region where tows are exposed
Individual tow MPUL measurement.
Measure fibre tension as a result of fibre relaxation and/or length differences
Measure sizing
   Content
   Quality
   Advancement of sizing
Measure resin advancement from compaction roller
Calculate RC by looking at the difference between test region and adjacent impregnated patch
Fibre surface characterisation
   Shine
   Entanglement
   Twist and ecta
Measure degree of fibre alignment to predict mechanical properties
Measure degree of homogeneity of tows overlap also to predict Cpt

Accordingly, in a second aspect, the invention relates to a method of testing a prepreg as described herein wherein the method comprises the steps of a) carrying out a measurement test on a test area of the test region, followed by b) inferring a property of the prepreg from the results of the measurement.

As discussed, the test region is particularly useful for measuring the fibre areal weight (FAW) of the prepreg. Accordingly, the method preferably comprises the steps of a) cutting out a test sample of fibres from a test area of the test region, b) weighing the test sample of fibres and c) calculating the weight per unit area of the test sample, and d) inferring that the fibre weight per unit are of the test sample is a measurement of the fibre weight per unit area of the prepreg.

The testing may be carried out whilst the prepreg is under tension, to reflect the tension experienced during manufacture, as appropriate to the test being carried out.

The prepregs according to the invention may be manufactured in known manner, typically in a continuous process involving the passage of many thousands of fibres, forming a structural layer of fibres, through a series of impregnation stages, typically guided by rollers, which act to impregnate resin into the structural layer. The point where the fibres meet the resin, usually in sheet form, is the start of the impregnation stage.

Thus, in a third aspect, the invention relates to a process for the manufacture of a prepreg as described herein, the process comprising the steps of: providing a structural layer comprising fibres, having a first face and a second face, and a first impregnating layer comprising curable thermosetting resin; bringing the first face of the structural layer into contact with the first impregnating layer; compressing the structural layer and first impregnation layer together so that curable resin impregnates the structural layer so that it is present between the interstices between the fibres, thereby forming the prepreg; wherein the first impregnating layer comprises a region where no impregnation occurs, thereby producing the test region of the prepreg that is free of curable resin.

Before the fibres are contacted with the resin and reach the impregnation zone they are typically arranged in a plurality of tows of unidirectional fibres, each tow comprising many thousands of filaments, e.g. 12,000. These tows are mounted on bobbins and are fed initially to a combing unit to ensure even separation of the fibres.

In order to improve handling of the resin it is conventional that it is supported onto a backing material, such as paper. The resin is then fed, typically from a roll, such that it comes into contact with the fibres, the backing material remaining in place on the exterior of the resin and fibre contact region. During the subsequent impregnation process the backing material provides a useful exterior material to apply pressure to, in order to achieve even impregnation of resin.

In one arrangement of the process, the region where no impregnation occurs is a region of the impregnating layer that is free of curable resin. For example, this may be achieved by a region of the backing material supporting the resin being free of resin. For example, this could be achieved by placing a label on the impregnating resin layer, and removing the label with resin transferred to it, in order to produce the region of the backing material that is free of resin.

In an alternative arrangement of the process, the region where no impregnation occurs is a region comprising a barrier sheet adhered to the first impregnating layer, that prevents impregnation of curable resin into the structural layer. Thus, resin may be present, but is prevented from migrating to the test region due to the presence of the barrier sheet. Preferably the barrier sheet comprises an adhesive to adhere it to the first impregnating layer, as this aids with its positioning and later removal.

Some resin may flow past the sides of the barrier sheet and therefore it should be appreciated that the test region may be a little smaller in dimension that the barrier sheet.

If difficulties are encountered with resin flowing past the sides of the barrier, then both solutions may be combined, i.e. preparing a region of the backing material that is free of resin, combined with placing a barrier sheet adhered to the backing material that is free of resin.

The barrier sheet may be made from a variety of materials, especially those that can withstand temperatures of 60 to 150° C. and can withstand the pressures of impregnation. Examples of suitable barrier sheets are vellum or polyethylene terephthalate (PET).

Typically, the process comprises the following step of removing the barrier sheet, to expose the test region of the prepreg for testing. This may involve cutting through any backing material supporting the resin.

Preferably a second impregnating layer comprising thermosetting resin is provided, wherein the second face of the fibrous layer is brought into contact with the second impregnating layer prior to the compressing, wherein the second impregnating layer comprises a region where no impregnation occurs, and arranged to be aligned with the region where no impregnation occurs in the first impregnating layer, thereby producing the test region of the prepreg that is free of curable resin.

To facilitate impregnation of the resin into the fibres it is conventional for this to be carried out at an elevated temperature, e.g. from 60 to 150° C. preferably from 100 to 130° C., so that the resin viscosity reduces. This is most conveniently achieved by heating the resin and fibres, before impregnation, to the desired temperature, e.g. by passing them through an infra-red heater. Following impregnation there is typically a cooling step, to reduce the tackiness of the formed prepreg. This cooling step can be used to identify the end of the impregnation stage.

This may be followed by further treatment stages such as laminating, slitting and separating.

Once prepared the prepreg may be rolled-up so that it can be stored for a period of time. It can then be unrolled and cut as desired. For example, testing may be carried out on a portion of the prepreg containing the test region, either before or after roll-up.

Once the prepregs are produced by the process of the present invention, a plurality of them are typically stacked together, producing a prepreg stack or preform. The prepreg stack or perform may then be cured by exposure to elevated temperature, wherein the thermosetting resin cures. This is typically carried out under elevated pressure in known manner such as the autoclave or vacuum bag techniques.

The invention will now be illustrated, by way of example only, with reference to the following FIGURE, in which:

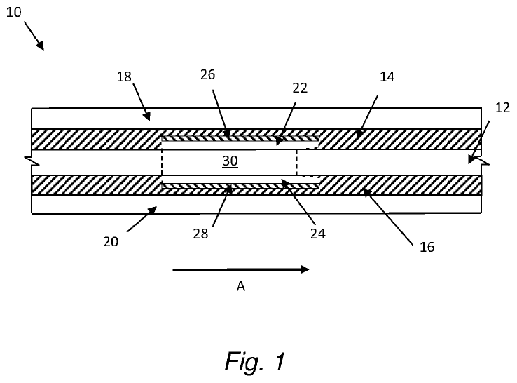

FIG. 1 is a side sectional view through a prepreg according to the present invention in the process of being manufactured.

FIG. 1 shows a prepreg 10 comprising a structural layer 12 comprising fibres and comprising a first impregnating layer 14 comprising curable thermosetting resin and a second impregnating layer 16 comprising curable thermosetting resin. The fibres are non-woven unidirectional carbon fibres aligned to be parallel with arrow A. The first impregnating layer 14 is supported on a first backing material 18, which is made of backing paper. The second impregnating layer 16 is supported on a second backing material 20 which is also made of backing paper.

Embedded within the first impregnating layer 14 is a first barrier sheet 22 made from vellum or PET. The first barrier sheet 22 comprises an adhesive 26 to adhere it to the first impregnating layer 14. Embedded within the second impregnating layer 16 is a second barrier sheet 24 made from vellum or PET. The second barrier sheet 26 comprises an adhesive 28 to adhere it to the second impregnating layer 16.

The prepreg 10 is shown shortly after a process for its manufacture has been carried out, in which the structural layer 12, the first impregnating layer 14 and the second impregnating layer 16 has been compressed by passing them over heated rollers under tension, travelling in direction of arrow A, such that a portion of the thermosetting resin impregnates the fibres in the structural layer 12, thereby forming the prepreg 10.

However, due to the presence of the first barrier sheet 22 and second barrier sheet 24, the prepreg comprises a region 30 where no impregnation occurs, thereby producing the test region 30 of the prepreg that is free of curable resin. It can be seen that, due to the temperatures and pressures involved in the formation of the prepreg, some curable resin passed underneath the first barrier sheet 22 and second barrier sheet 24, such that the test region 30 is smaller than the size of the first barrier sheet 22 and second barrier sheet 24.

Testing of the prepreg in the test region may be carried out, possibly even before the prepreg manufacturing process is completed, as discussed above.

### EXAMPLES

Prepregs were manufactured, in the manner shown in FIG. 1, by bringing together a layer of unidirectional carbon fibres with an upper resin sheet on backing paper and a lower resin sheet also on backing paper. The three layers were brought together by passing over and between heated rollers, in known manner, in order for the resin to impregnate the carbon fibres into the interstices between the fibres.

A sheet of vellum of dimension 125 mm×197 mm with an acrylic resin adhesive on one face was placed on the upper resin sheet, so that the sheet of vellum prevents any resin from passing and entering the carbon fibres. A sheet of vellum of the same size was placed on the lower resin sheet, and aligned with the sheet of vellum on the upper resin sheet.

Thus, as the prepreg was formed, a test region that is free of curable thermosetting resin with dimensions approximately 125 mm×197 mm was left in the prepreg. The prepreg comprising the test region was rolled up and stored. The roll of prepreg was passed to an editing machine, and unrolled under tension, to expose the test region for testing.

A region of 100 mm×100 mm was cut out of the test region and weighed, to determine the fibre areal weight (FAW). On a region 1 m apart from the test region, a conventional washout test according to ASTM D3529 was also performed to determine the fibre areal weight (FAW). The results are shown in table 1 below, with the values shown in gsm.

### Table 1

TABLE 1

| Sample No. | Dry Fiber FAW | Washout FAW |
|---|---|---|
| 1 | 188.5 | 188.9 |
| 2 | 189.8 | 187.1 |
| 3 | 188.5 | 189.1 |
| 4 | 191 | 190.8 |
| 5 | 185.9 | 189.4 |
| 6 | 187 | 187.5 |
| 7 | 184.8 | 193.1 |

It can be seen that the measurement of FAW from the test region was highly consistent with the washout method.

The procedure was repeated at a later date and the results are shown in table 2 below, with the values shown in gsm which also show the prepreg areal weight, PAW.

TABLE 2

| Sample No. | Dry Fiber FAW | PAW | Washout FAW |
|---|---|---|---|
| 1 | 271.5 | 409 | 272.2 |
| 2 | 274 | 412 | 275.9 |
| 3 | 273.7 | 410.4 | 273.5 |
| 4 | 272.8 | 413.6 | 275.1 |
| 5 | 271.5 | 412.4 | 276.1 |
| 6 | 272 | 415.2 | 275.7 |
| 7 | 273.7 | 408.1 | 271.3 |
| 8 | 273.7 | 414.9 | 273.9 |
| 9 | 270.2 | 415 | 276.7 |
| 10 | 267.6 | 410.4 | 275.7 |
| 11 | 267.7 | 411.9 | 275.9 |
| 12 | 272.1 | 415 | 277.4 |
| 13 | 271.9 | 417.9 | 273.4 |
| 14 | 273.2 | 416.9 | 275 |
| 15 | 268.4 | 412.9 | 275.2 |
| 16 | 270.2 | 412.7 | 273.9 |
| 17 | 271.2 | 411.3 | 273.7 |
| 18 | 266.2 | 416.6 | 274.1 |
| 19 | 271.1 | 415.3 | 274.6 |
| 20 | 266.2 | 417.4 | 274.1 |
| 21 | 268.1 | 411 | 269.5 |
| 22 | 268.1 | 411.7 | 272.8 |
| 23 | 266.5 | 412.2 | 275.6 |
| 24 | 267.6 | 412 | 274.4 |
| 25 | 267.3 | 412.6 | 271.1 |
| 26 | 271.5 | 409 | 272.2 |

It can be seen that the measurement of FAW from the test region was highly consistent with the washout method.

The procedure was repeated at a later date, although this time taking many more samples, and from different positions on the prepreg, and the results are shown in table 3 below, with the values shown in gsm which also show the prepreg areal weight, PAW, and Resin Content (RC).

TABLE 3

| Sample | | Washout | Dry Fiber | | |
|---|---|---|---|---|---|
| No. | PAW | FAW | FAW | ΔFAW | RC |
| 1 | 288.05 | 190.91 | 190.24 | 0.67 | 33.72% |
| 2 | 289.39 | 191.54 | 189 | 2.54 | 33.81% |
| 3 | 291.06 | 192.49 | 189.64 | 2.85 | 33.87% |
| 4 | 289.52 | 191.66 | 190.96 | 0.70 | 33.80% |
| 5 | 291.52 | 191.45 | 190.28 | 1.17 | 34.33% |
| 6 | 293.12 | 193.12 | 193.75 | −0.63 | 34.12% |
| 7 | 292.88 | 193.55 | 190.17 | 3.38 | 33.91% |
| 8 | 293.99 | 193.2 | 191.05 | 2.15 | 34.28% |
| 9 | 290.9 | 192.16 | 191.62 | 0.54 | 33.94% |
| 10 | 293.1 | 192.59 | 191.47 | 1.12 | 34.29% |
| 11 | 289.12 | 191.1 | 191.84 | −0.74 | 33.90% |
| 12 | 289.15 | 193.33 | 191.47 | 1.86 | 33.14% |
| 13 | 290.88 | 192.44 | 190.08 | 2.36 | 33.84% |
| 14 | 293.17 | 192.42 | 188.49 | 3.93 | 34.37% |
| 15 | 287.95 | 190.47 | 190.76 | −0.29 | 33.85% |
| 16 | 289.81 | 191.08 | 190.98 | 0.10 | 34.07% |
| 17 | 293.77 | 193.65 | 191.1 | 2.55 | 34.08% |
| 18 | 292.01 | 193.19 | 193.39 | −0.20 | 33.84% |
| 19 | 290.4 | 193.13 | 191.78 | 1.35 | 33.50% |
| 20 | 293.8 | 192.57 | 191.73 | 0.84 | 34.46% |
| 21 | 291.68 | 192.15 | 192.33 | −0.18 | 34.12% |
| 22 | 291.69 | 192.32 | 192.79 | −0.47 | 34.07% |
| 23 | 289.72 | 191.15 | 191.03 | 0.12 | 34.02% |
| 24 | 292.19 | 193.41 | 192.23 | 1.18 | 33.81% |
| 25 | 290.45 | 192.15 | 189.53 | 2.62 | 33.84% |
| 26 | 291.63 | 193.01 | 190.99 | 2.02 | 33.82% |
| 27 | 288.14 | 192.62 | 189.2 | 3.42 | 33.15% |
| 28 | 289.82 | 193.69 | 194.45 | −0.76 | 33.17% |
| 29 | 283.91 | 190.57 | 190.32 | 0.25 | 32.88% |
| 30 | 293.79 | 192.66 | 190.19 | 2.47 | 34.42% |
| 31 | 292.84 | 193.55 | 193.34 | 0.21 | 33.91% |
| 32 | 294.2 | 194.45 | 192.28 | 2.17 | 33.91% |
| 33 | 292.21 | 193.66 | 192.08 | 1.58 | 33.73% |
| 34 | 290.88 | 191.88 | 192.11 | −0.23 | 34.03% |
| 35 | 291.63 | 192.36 | 191.53 | 0.83 | 34.04% |
| 36 | 289.98 | 190.7 | 191.9 | −1.20 | 34.24% |
| 37 | 292.19 | 193.45 | 190.62 | 2.83 | 33.79% |
| 38 | 290.79 | 192.63 | 192.11 | 0.52 | 33.76% |
| 39 | 290.61 | 191.09 | 190.56 | 0.53 | 34.25% |
| 40 | 286.84 | 191.9 | 191.52 | 0.38 | 33.10% |
| 41 | 287.5 | 189.63 | 191.4 | −1.77 | 34.04% |
| 42 | 290.05 | 192.31 | 192.28 | 0.03 | 33.70% |
| 43 | 297.58 | 193.26 | 192.81 | 0.45 | 35.06% |
| 44 | 291.48 | 193.29 | 191.85 | 1.44 | 33.69% |
| 45 | 289.54 | 190.77 | 190.76 | 0.01 | 34.11% |
| 46 | 292.59 | 193.51 | 191.16 | 2.35 | 33.86% |
| 47 | 289.96 | 191.72 | 193.99 | −2.27 | 33.88% |
| 48 | 292.01 | 193.29 | 191.97 | 1.32 | 33.81% |
| 49 | 287.76 | 191.07 | 191.4 | −0.33 | 33.60% |
| 50 | 289.98 | 192.75 | 190.33 | 2.42 | 33.53% |
| 51 | 291.74 | 192.49 | 188.78 | 3.71 | 34.02% |
| 52 | 290.64 | 192.62 | 190.69 | 1.93 | 33.73% |

It can be seen that the measurement of FAW from the test region was highly consistent with the washout method. The average FAW for the ‘dry’ test carried out on the test region was 191.31 and the average FAW for the washout test was 192.35. It may be speculated that this systematic difference in measurement may be due to residual resin being left behind in the washout method.

The procedure was repeated at a later date, although this time a region of 300 mm×300 mm was cut out of the test region and weighed, to determine the fibre areal weight (FAW), and the results are shown in table 3 below.

TABLE 4

| Sample No. | Washout FAW | Dry Fiber FAW | ΔFAW |
|---|---|---|---|
| 1 | 267.99 | 268.05 | 0.05 |
| 2 | 268.12 | 268.20 | 0.08 |
| 3 | 267.67 | 268.08 | 0.40 |
| 4 | 267.84 | 267.28 | 0.55 |
| 5 | 267.64 | 267.73 | 0.09 |
| 6 | 268.06 | 268.05 | 0.01 |
| 7 | 268.51 | 268.27 | 0.24 |
| 8 | 268.19 | 268.44 | 0.25 |
| 9 | 268.56 | 268.80 | 0.24 |
| 10 | 267.79 | 267.47 | 0.32 |

It can be seen that the increased size of the test region from 100 mm×100 mm to 300 mm×300 mm did not have a significant effect.

1. A prepreg comprising a structural layer comprising fibres having interstices therebetween, and comprising curable thermosetting resin impregnated within the structural layer and present within the interstices, wherein the prepreg comprises a test region that is free of curable thermosetting resin; wherein the test region has a surface area of from 50 to 500 cm2; wherein the test region is surrounded by regions of the prepreg that are impregnated with curable thermosetting resin; wherein said regions of the prepreg impregnated with curable resin comprise 25 to 50 wt. % of curable resin; wherein the fibres are discrete and not interwoven; wherein the fibres are unidirectional; wherein the fibres are carbon or glass fibres.

2. (canceled)

3. (canceled)

4. (canceled)

5. The prepreg according to claim 1, which comprises a second test region that is free of curable thermosetting resin.

6. The prepreg according to claim 5, which comprises a third test region that is free of curable thermosetting resin.

7. (canceled)

8. (canceled)

9. (canceled)

10. (canceled)

11. The method of testing a pregreg according to claim 1, wherein the method comprises the steps of a) carrying out a measurement test on a test area of the test region, followed by b) inferring a property of the prepreg from the results of the measurement.

12. The method of testing a prepreg according to claim 11, wherein the method comprises the steps of a) cutting out a test sample of fibres from a test area of the test region, b) weighing the test sample of fibres and c) calculating the weight per unit area of the test sample, and d) inferring that the fibre weight per unit are of the test sample is a measurement of the fibre weight per unit area of the prepreg.

13. The method of testing according to claim 11, wherein the testing is carried out while the prepreg is under tension comparable to the tension experienced during manufacture of the prepreg.

14. (canceled)

15. (canceled)

16. (canceled)

17. (canceled)

18. (canceled)

19. (canceled)

20. (canceled)

21. (canceled)

22. (canceled)
